(19) United States
(12) Patent Application Publication
(10) Pub. No.: US 2016/0354230 A1
(43) Pub. Date: Dec. 8, 2016

(54) Female Urination Aid

(71) Applicant: Muhammad Abuhaikal, Bronx, NY (US)

(72) Inventor: Muhammad Abuhaikal, Bronx, NY (US)

(21) Appl. No.: 15/086,872

(22) Filed: Mar. 31, 2016

Related U.S. Application Data
(60) Provisional application No. 62/172,314, filed on Jun. 8, 2015.

(51) Int. Cl. A61F 5/455

(52) U.S. Cl. CPC A61F 5/4556

(57) ABSTRACT

A female urination aid allowing hygienic upright urination includes a ring. A shell is coupled to an inner perimeter of the ring. The shell is collapsible and has a bottom, which is open. The bottom has a circumference that is dimensionally smaller than the inner perimeter, such that the shell is funnel shaped. A grasp is coupled to the shell proximate to the bottom. The ring is configured for sealable placement around a vulva of a user. The grasp is configured for grasping by the user to deploy the shell from a collapsed positioned to an extended position. The bottom of the shell is distally positioned relative to the ring, such that the shell is configured to direct urine away from the user.

### CROSS-REFERENCE TO RELATED APPLICATIONS

This application claims benefit of the provisional application Ser. No. 62/172,314 filed Jun. 8, 2015.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIE THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

(1) Field of the Invention

(2) Description of Related Art Including Information Disclosed Under 37 CFR 1.97 and 1.98

The disclosure and prior art relates to urination aids and more particularly pertains to a new urination aid for females allowing hygienic upright urination.

### BRIEF SUMMARY OF THE INVENTION

An embodiment of the disclosure meets the needs presented above by generally comprising a ring. A shell is coupled to an inner perimeter of the ring. The shell is collapsible and has a bottom, which is open. The bottom has a circumference that is dimensionally smaller than the inner perimeter, such that the shell is funnel shaped. A grasp is coupled to the shell proximate to the bottom. The ring is configured for sealable placement around a vulva of a user. The grasp is configured for grasping by the user to deploy the shell from a collapsed positioned to an extended position. The bottom of the shell is distally positioned relative to the ring, such that the shell is configured to direct urine away from the user.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric perspective view of a female urination aid according to an embodiment of the disclosure.

FIG. 2 is a back view of an embodiment of the disclosure.

FIG. 3 is a top view of an embodiment of the disclosure.

FIG. 4 is a side view of an embodiment of the disclosure.

FIG. 5 is an in-use view of an embodiment of the disclosure.

### DETAILED DESCRIPTION OF THE INVENTION

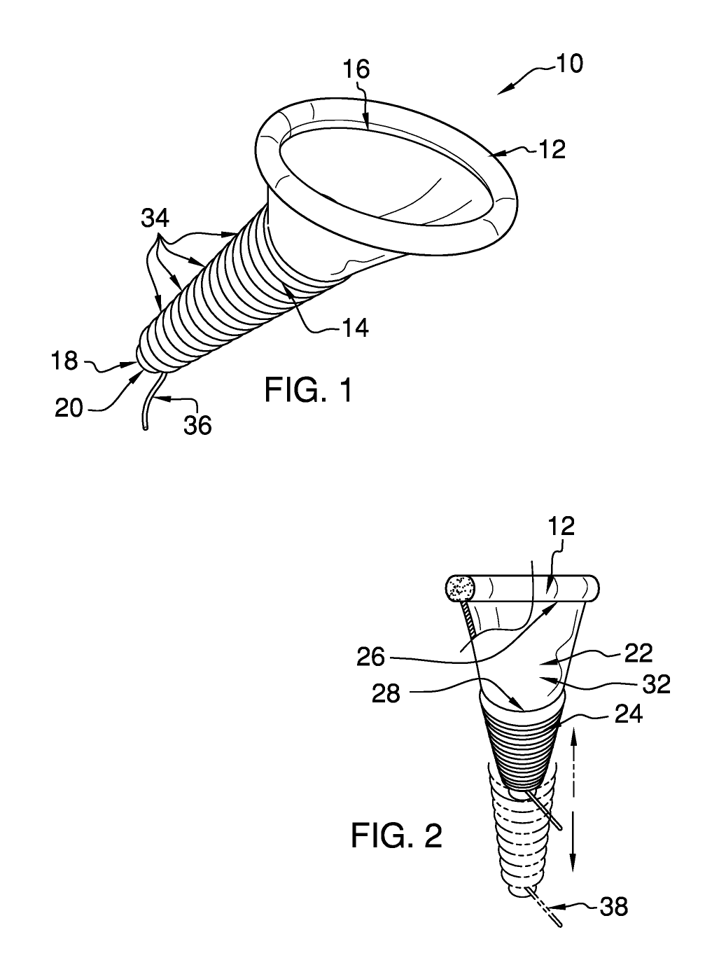

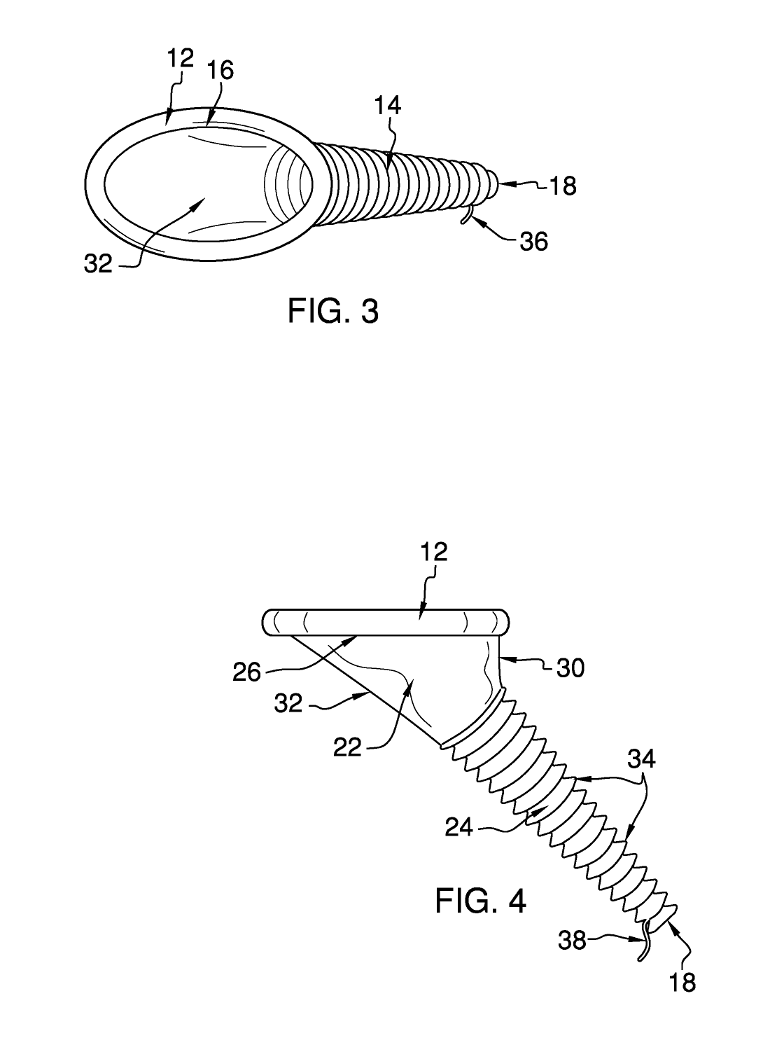

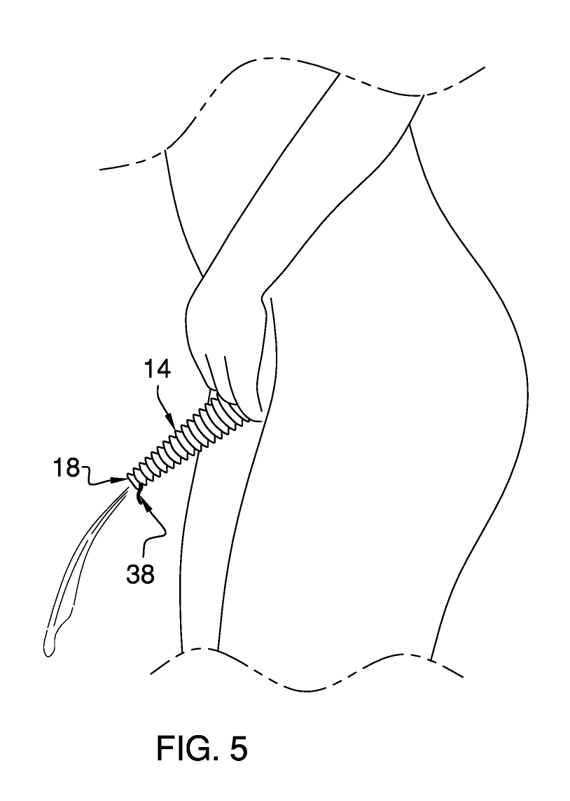

With reference now to the drawings, and in particular to FIGS. 1 through 5 thereof, a new urination aid embodying the principles and concepts of an embodiment of the disclosure and generally designated by the reference numeral 10 will be described.

As best illustrated in FIGS. 1 through 5, the female urination aid 10 generally comprises a ring 12 that is ovally shaped. The ring 12 is pliable, such that the ring 12 is configured to conform to a vulvar region of a user. In one embodiment, the ring 12 comprises foam.

A shell 14 is coupled to an inner perimeter 16 of the ring 12. The shell 14 has a bottom 18, which is open. The bottom 18 has a circumference 20 that is dimensionally smaller than the inner perimeter 16, such that the shell 14 is funnel shaped. The shell 14 is collapsible.

The shell 14 comprises an upper section 22 and a lower section 24. The upper section 22 has a top end 26 and a bottom end 28. The top end 26 is coupled to the ring 12. The top end 26 is cross-sectionally ovally shaped. The bottom end 28 is cross-sectionally circularly shaped. A top face 30 of the upper section 22 extends substantially perpendicularly from the ring 12 and a bottom face 32 of the upper section 22 extends transversely from the ring 12. The lower section 24 is coupled to and extends from the bottom end 28 of the upper section 22 defining the bottom 18 of the shell 14. The lower section 24 is cross-sectionally circularly shaped. In one embodiment, the lower section 24 comprises a plurality of pleats 34.

The shell 14 and the ring 12 are water resistant. The shell 14 and the ring 12 comprise paper, such that the shell 14 and the ring 12 are biodegradable.

A grasp 36 is coupled to the shell 14 proximate to the bottom 18. The grasp 36 comprises a cord 38.

In use, the ring 12 is configured for sealable placement around a vulva of the user. The grasp 36 is positioned for grasping by the user for deployment of the shell 14 from a collapsed positioned to an extended position. In the extended position, the bottom 18 of the shell 14 is distally positioned relative to the ring 12, such that the shell 14 is configured to direct urine away from the user.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded.

A reference to an element by the indefinite article “a” does not exclude the possibility that more than one of the element is present, unless the context clearly requires that there be only one of the elements.

I claim:

1. A female urination aid comprising:
a ring;
a shell coupled to an inner perimeter of said ring, said shell having a bottom, said bottom being open, said bottom having a circumference, said circumference being dimensionally smaller than said inner perimeter, wherein said shell is funnel shaped, said shell being collapsible;
a grasp coupled to said shell proximate to said bottom; and
wherein said ring is configured for sealable placement around a vulva of the user, wherein said grasp is configured for grasping by the user for deployment of said shell from a collapsed positioned to an extended position wherein said bottom of said shell is distally positioned relative to said ring, such that said shell is configured to direct urine away from the user.

2. The aid of claim 1, further including said ring being ovally shaped.

3. The aid of claim 1, further including said ring being pliable, such that said ring is configured to conform to a vulvar region of the user.

4. The aid of claim 3, further including said ring comprising foam.

5. The aid of claim 1, further including said shell comprising:
an upper section, said upper section having a top end and a bottom end, said top end being coupled to said ring, wherein a top face of said upper section extends substantially perpendicularly from said ring and a bottom face of said upper section extends transversely from said ring; and
a lower section, said lower section being coupled to and extending from said bottom end of said upper section defining said bottom of said shell.

6. The aid of claim 5, further comprising:
said top end being cross-sectionally ovally shaped;
said bottom end being cross-sectionally circularly shaped; and
said lower section being cross-sectionally circularly shaped.

7. The aid of claim 6, further including said lower section comprising a plurality of pleats.

8. The aid of claim 1, further including said shell and said ring being water resistant.

9. The aid of claim 8, further including said shell and said ring comprising paper, such that said shell and said ring are biodegradable.

10. The aid of claim 1, further including said grasp comprising a cord.

11. A female urination aid comprising:
a ring, said ring being ovally shaped, said ring being pliable, such that said ring is configured to conform to a vulvar region of a user, said ring comprising foam;
a shell coupled to an inner perimeter of said ring, said shell having a bottom, said bottom being open, said bottom having a circumference, said circumference being dimensionally smaller than said inner perimeter, wherein said shell is funnel shaped, said shell being collapsible, said shell comprising:
an upper section, said upper section having a top end and a bottom end, said top end being coupled to said ring, said top end being cross-sectionally ovally shaped, said bottom end being cross-sectionally circularly shaped, wherein a top face of said upper section extends substantially perpendicularly from said ring and a bottom face of said upper section extends transversely from said ring,
a lower section, said lower section being coupled to and extending from said bottom end of said upper section defining said bottom of said shell, said lower section being cross-sectionally circularly shaped, said lower section comprising a plurality of pleats;
said shell and said ring being water resistant, said shell and said ring comprising paper, such that said shell and said ring are biodegradable;
a grasp coupled to said shell proximate to said bottom, said grasp comprising a cord; and
wherein said ring is configured for sealable placement around a vulva of the user, wherein said grasp is configured for grasping by the user for deployment of said shell from a collapsed positioned to an extended position wherein said bottom of said shell is distally positioned relative to said ring, such that said shell is configured to direct urine away from the user.
